SEQUENCE LISTING (24 sequences)

SEQ ID NO: 1 - RNA, 22 nt, unidentified
  source 1..22: mol_type = other RNA
tccctgagaccctaacttgtga

SEQ ID NO: 2 - RNA, 22 nt, unidentified
  source 1..22: mol_type = other RNA
tccctgagaccctaacttgaga

SEQ ID NO: 3 - RNA, 22 nt, unidentified
  source 1..22: mol_type = other RNA
tcgctgagaccctaacttgtga

SEQ ID NO: 4 - RNA, 22 nt, unidentified
  source 1..22: mol_type = other RNA
tccctgagaccgtaacttgtga

SEQ ID NO: 5 - RNA, 22 nt, unidentified
  source 1..22: mol_type = other RNA
tagcagcacataatggtttgtg

SEQ ID NO: 6 - RNA, 22 nt, unidentified
  source 1..22: mol_type = other RNA
tagcagcacataatggtttctg

SEQ ID NO: 7 - RNA, 22 nt, unidentified
  source 1..22: mol_type = other RNA
taccagcacataatggtttgtg

SEQ ID NO: 8 - RNA, 22 nt, unidentified
  source 1..22: mol_type = other RNA
tagcagcacattatggtttgtg

SEQ ID NO: 9 - RNA, 22 nt, unidentified
  source 1..22: mol_type = other RNA
ttatcagaatctccaggggtac

SEQ ID NO: 10 - RNA, 22 nt, unidentified
  source 1..22: mol_type = other RNA
ttatcagaatctccaggggaac

SEQ ID NO: 11 - RNA, 22 nt, unidentified
  source 1..22: mol_type = other RNA
ttctcagaatctccaggggtac

SEQ ID NO: 12 - RNA, 22 nt, unidentified
  source 1..22: mol_type = other RNA
ttatcagaatcaccaggggtac

SEQ ID NO: 13 - DNA, 11 bp, unidentified
  source 1..11: mol_type = other DNA
tcacaagttag

SEQ ID NO: 14 - DNA, 11 bp, unidentified
  source 1..11: mol_type = other DNA
cacaaaccatt

SEQ ID NO: 15 - DNA, 11 bp, unidentified
  source 1..11: mol_type = other DNA
gtacccctgga

SEQ ID NO: 16 - DNA, 11 bp, unidentified
  source 1..11: mol_type = other DNA
ggtctcaggga

SEQ ID NO: 17 - DNA, 11 bp, unidentified
  source 1..11: mol_type = other DNA
atgtgctgcta

SEQ ID NO: 18 - DNA, 11 bp, unidentified
  source 1..11: mol_type = other DNA
gattctgataa

SEQ ID NO: 19 - DNA, 11 bp, unidentified
  source 1..11: mol_type = other DNA
tcacaagttag

SEQ ID NO: 20 - DNA, 11 bp, unidentified
  source 1..11: mol_type = other DNA
cacaaaccatt

SEQ ID NO: 21 - DNA, 11 bp, unidentified
  source 1..11: mol_type = other DNA
gtacccctgga

SEQ ID NO: 22 - DNA, 11 bp, unidentified
  source 1..11: mol_type = other DNA
ggtctcaggga

SEQ ID NO: 23 - DNA, 11 bp, unidentified
  source 1..11: mol_type = other DNA
atgtgctgcta

SEQ ID NO: 24 - DNA, 11 bp, unidentified
  source 1..11: mol_type = other DNA
gattctgataa

(19) United States
(12) Patent Application Publication
(10) Pub. No.: US 2023/0193365 A1
(43) Pub. Date: Jun. 22, 2023

(54) METAL NANOSTRUCTURE BASED ON BIOMOLECULES AND NANOPLASMONIC BIOSENSOR USING THE SAME

(71) Applicant: Korea University Research and Business Foundation, Seoul (KR)

(72) Inventors: Sang Jun SIM, Seoul (KR); So Jin SONG, Seoul (KR); Jong Uk LEE, Seoul (KR)

(73) Assignee: Korea University Research and Business Foundation, Seoul (KR)

(21) Appl. No.: 17/869,856

(22) Filed: Jul. 21, 2022

(30) Foreign Application Priority Data
Jul. 21, 2021 (KR) 10-2021-0096028
Jul. 5, 2022 (KR) 10-2022-0082508

(51) Int. Cl. C12Q 1/6834; C12Q 1/6883; C12Q 1/6825

(52) U.S. Cl. CPC C12Q 1/6834; C12Q 1/6883; C12Q 1/6825; C12Q 2600/178

(57) ABSTRACT

Disclosed are a metal nanostructure based on biomolecules and a nanoplasmonic biosensor using the same.

### CROSS-REFERENCE TO RELATED APPLICATIONS

This application claims the benefit under 35 USC § 119(a) of Korean Patent Application No. 10-2021-0096028 filed on Jul. 21, 2021 and Korean Patent Application No. 10-2022-00082508 filed on Jul. 5, 2022 in the Korean Intellectual Property Office, the entire disclosure of which is incorporated herein by reference for all purposes.

### INCORPORATION-BY-REFERENCE OF MATERIAL SUBMITTED BY U.S.P.T.O. EFS-WEB

This application contains a Sequence Listing, which is being submitted in computer readable form via the United States Patent and Trademark Office Patent Center and which is hereby incorporated by reference in its entirety for all purposes. The XML file submitted herewith, which is named as “NIA20221215_0181590036_SequenceListing” and is created on Dec. 15, 2022, contains a 21.5 KB file.

### BACKGROUND OF THE INVENTION

### Field of the Invention

The present invention relates to a metal nanostructure based on biomolecules and a nanoplasmonic biosensor using the same.

### Description of the Related Art

Recent statistics have shown that about 50 million people worldwide suffer from dementia and this number is expected to double every 20 years and thus reach about 152 million cases by 2050. Thereamong, Alzheimer's disease (AD) is the most common neurodegenerative disease in the elderly, characterized by progressive cognitive and functional decline, and its main symptoms are memory loss and inability to create new memories. In particular, when Alzheimer's disease progresses beyond a certain level, the progression thereof can only be slowed and the disease is irreversible. Therefore, early diagnosis and recognition of symptoms are very important.

It has been reported that Alzheimer's disease is caused by various complicated pathological and physiological conditions such as amyloid cascades, tau phosphorylation, neurotransmitters, excitotoxicity or oxidative stress. Accordingly, it is very difficult to prevent the onset or progression of Alzheimer's disease and thus the focus has now shifted to early diagnosis and treatment to slow the progression and ameliorate cognitive decline.

Alzheimer's disease (AD) has major pathological features of cerebral accumulation of β-amyloid (Aβ) peptides or neurofibrillary tangles (NFT) of tau, and is closely related to neurodegenerative mechanisms leading to toxicity and destruction of neurons and synapses during pathogenesis thereof.

There are current diagnostic methods such as brain imaging, cognitive function testing, and diagnostic tests using cerebrospinal fluid. However, these methods are inapplicable after the onset of most of the symptoms of Alzheimer's disease and thus are insufficient for early diagnosis. In addition, these methods have a limitation in that tests based on these biomarkers are not always feasible due to, for example, the economic drawback of entailing a high diagnostic cost and the potential risk of invasive procedures.

Meanwhile, blood-based diagnostics can help overcome these drawbacks since they are non-invasive, inexpensive, and allow multiple sampling even at large scales. Accordingly, many recent studies regarding the correlation between blood-based Alzheimer's disease biomarkers and pathological changes in the brain have been reported. However, despite these efforts, a blood-based Alzheimer's disease biomarker-based test method that can directly reflect the onset and progression of Alzheimer's disease still remains insufficient.

Meanwhile, exosomes are small vesicles having a size ranging from 30 to 100 nm that are secreted from various types of cells, including the central nervous system (CNS), and are reported to function to diffuse pathogenic proteins and facilitate the aggregation of proteins such as Aβ, tau and prions in the brain. In addition, exosomes can pass through the blood-brain barrier (BBB) while carrying various genetic materials (such as DNA, miRNA and proteins) related to nerve function, and thus information about the brain state can be obtained more easily therefrom. In particular, exosomal miRNAs (exo-miRs) are involved in various basic processes of the central nervous system, such as neuronal differentiation, development, and function of mature neurons, and thus provide accurate information on various characteristics of Alzheimer's disease according to disease progression. Therefore, exo-miR has been highlighted as a novel diagnostic and therapeutic biomarker for AD. However, the concentration of blood-based biomarkers is 10-10² times lower than the concentration thereof in cerebrospinal fluid and the blood also contains many other interfering substances, so the development of accurate and reliable methods to detect these biomarkers is required.

Furthermore, recently, exo-miR has been detected using quantitative reverse transcription polymerase chain reaction (qRT-PCR) and microarray methods, which are mainly based on fluorescence, and thus have limitations of reduced detection reliability and sensitivity due to problems associated with fluorescent dyes (e.g., photobleaching and flickering). In addition, these methods have limitation of difficulty in realizing accurate analysis due to the characteristics of miRNA (e.g., short length in body fluids, high homology, and low expression level).

Recently, plasmonic single nanoparticle (NP)-based sensors have gained increasing interest as an alternative to overcome these limitations based on the ability thereof to interact with incident light to generate localized surface plasmon resonance (LSPR). This physical phenomenon allows for super-sensitivity to even single NP with a small size by absorbing the change in the refractive index (RI) around the NP and converting the same into the shift of the plasmonic band in the scattering spectrum. In addition, since the LSPR greatly depends on the shape, size, local RI, and arrangement of nanoparticles, the sensitivity of the nanoparticle-based LSPR biosensors can be easily improved by the adjustment of these factors. In keeping with this trend, several recent studies have introduced LSPR-based sensors for detecting miRNA. However, the use of exo-miR in clinical applications is still limited due to insufficient detection sensitivity, and low reproducibility and selectivity thereof. Therefore, there is a need for research to overcome these limitations.

### SUMMARY OF THE INVENTION

Therefore, the present invention has been made in view of the above problems, and it is one object of the present invention to provide a label-free plasmonic biosensor based on a DNA-assembled advanced plasmonic architecture (DAPA) and a method of accurately and simultaneously detecting an exo-miR biomarker of Alzheimer's disease in isolated serum at a very low detection limit using the same.

In accordance with the present invention, the above and other objects can be accomplished by the provision of a single metal-DAPA.

In an embodiment, the metal-DAPA may include at least one metal nanosphere conjugated with one single-stranded DNA (1ssDNA), and at least one metal nanosphere conjugated with two single-stranded DNAs (2ssDNA) complementary thereto, wherein the metal nanospheres are bridged to one another and the single metal-DAPA comprises a nanogap greater than 0 and not greater than 2 nm.

In another embodiment, the DAPA may include the metal nanosphere conjugated with one single-stranded DNA (1ssDNA) and the metal nanosphere conjugated with two single-stranded DNAs (2ssDNA) complementary thereto at a ratio of 2:1.

In another embodiment, the metal may include any one selected from the group consisting of gold (Au), copper (Cu), platinum (Pt) and palladium (Pd).

In another embodiment, the single metal-DAPA may be used for a biosensor.

In accordance with another aspect of the present invention, provided is a single DAPA-based label-free nanoplasmonic biosensor.

In an embodiment, the biosensor may include a substrate, a metal-DAPA (DNA-assembled advanced plasmonic architecture) fixed to the substrate, the metal-DAPA conjugated with a capture probe specifically binding to a target biomarker, and a measuring device configured to measure localized surface plasmon resonance in the metal-DAPA.

In another embodiment, the metal-DAPA may include three metal nanospheres conjugated with single-stranded DNA and bridged to one another.

In another embodiment, the DAPA may include at least one metal nanosphere conjugated with one single-stranded DNA (1ssDNA) and at least one metal nanosphere conjugated with two single-stranded DNAs (2ssDNA) complementary thereto at a ratio of 2:1.

In another embodiment, the DNA may have a length of 75 bp to 150 bp.

In another embodiment, the metal-DAPA may include a nanogap.

In another embodiment, the nanogap may be greater than 0 nm and not greater than 2 nm.

In another embodiment, the metal may include any one selected from the group consisting of gold (Au), copper (Cu), platinum (Pt) and palladium (Pd).

In another embodiment, the target biomarker may be DNA, miRNA or peptide.

In another embodiment, the miRNA may be derived from an exosome.

In another embodiment, the miRNA may be exo-miR125b, exo-miR15a, exo-miR361, or a combination thereof.

In another embodiment, the biosensor may detect exosome-derived miRNA proteins by measuring a change in Rayleigh scattering spectrum caused by specific binding of exosome-derived miRNA.

In another embodiment, the capture probe may include DNA or LNA.

In accordance with another aspect of the present invention, provided is a method of detecting exosome-derived miRNA including treating the biosensor with a biomarker mixture.

In an embodiment, the biomarker mixture may be blood or agglutinin-free serum.

In another embodiment, the biomarker mixture may include exosome-derived miRNA.

In accordance with another aspect of the present invention, provided is a method of diagnosing Alzheimer's disease including treating the biosensor with a biomarker mixture, and treating the biosensor with an exosome-derived miRNA detection probe.

In an embodiment, the detection probe may include DNA or LNA.

In accordance with another aspect of the present invention, provided is a method of preparing a single DAPA-based nanoplasmonic biosensor.

In an embodiment, the method may include (a) hybridizing single-stranded DNA-metal nanospheres with single-stranded DNA-metal nanospheres having a sequence complementary thereto at a ratio of 2:1 to obtain metal-DAPA seeds, (b) coating the metal-DAPA seeds with a predetermined material, (c) crystallizing the coated metal-DAPA seeds by treatment with a metal precursor and a reducing agent, (d) fixing the metal-DAPA to a substrate, and (e) conjugating the metal-DAPA with a capture probe specifically binding to an isolated target biomarker.

In another embodiment, the metal-DAPA seeds may be coated with polyethylene glycol (PEG).

In another embodiment, the method may further include coating the substrate with (3-mercaptopropyl)trimethoxysilane (MPTES) before step (d).

In another embodiment, the crystallization may be performed at a pH of 5.

In accordance with another aspect of the present invention, provided is a method of preparing a single metal-DAPA.

In an embodiment, the method may include (a) hybridizing one single-stranded DNA (1ssDNA)-metal nanospheres with two single-stranded DNAs (2ssDNA)-metal nanospheres having a sequence complementary thereto at a ratio of 2:1 to obtain metal-DAPA seeds, (b) coating the metal-DAPA seeds with a predetermined material, and (c) crystallizing the coated metal-DAPA seeds by treatment with a metal precursor and a reducing agent.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects, features, and other advantages of the present invention will be more clearly understood from the following detailed description taken in conjunction with the accompanying drawings, in which:

FIG. 1 illustrates the synthesis based on each design of a DNA-assembled advanced plasmon architecture (DAPA) according to an embodiment of the present invention. Specifically, (A) illustrates a designed gold nanoparticle (AuNP) architecture (top panel, unit: nm) and distribution of plasmon resonance electric field (bottom panel, unit: Vm⁻¹), (B) illustrates a localized surface plasmon resonance (LSPR) wavelength shift around AuNPs and a linear fit to the change in refractive index (RI), (C) is a schematic diagram illustrating the crystallization of Au atoms by reduction of AuCl₄⁻ with NH₃OH⁺ using a direction-specific crystallization method, and (D) illustrates DAPAs synthesized at a pH of 4 and at a pH of 5. The nanogap and nanoarchitecture were formed as shown in the EF-TEM image (scale bar, 5 nm). (E) is FE-TEM images (scale bars, 5 nm) illustrating DNA-induced crystallization of the box marked with white dotted lines (left) and fast Fourier transform patterns (right);

FIG. 2 shows the results of FDTD simulation of the Au-bridged nanoprobe according to an embodiment of the present invention. Specifically, (A) illustrates the designed Au bridge nanoprobe (top panel, unit: nm) and the distribution of the plasmon resonance electric field (bottom panel, unit: Vm⁻¹), (B) illustrates a localized surface plasmon resonance (LSPR) in the presence of the Au bridged nanoprobe and the linear fit to the change in refractive index (RI);

FIG. 3 illustrates an overall process for seeds of a DAPA according to an embodiment of the present invention. Specifically, (A) shows the results of separation of one single-stranded DNA (1ssDNA)-conjugated gold nanoparticles (AuNP) and two single-stranded DNAs (2ssDNA)-conjugated gold nanoparticles (AuNP) using gel electrophoresis, (B) shows the result of hybridization of 1DNA-AuNP and 2DNA-AuNP in a volume ratio of 2:1, and (C) shows the result of separation of DAPA seeds using gel electrophoresis;

FIG. 4 shows the size distribution of the DAPA according to an embodiment of the present invention. Specifically, (A), (B), (C) and (D) show the length, diameter, bridge diameter and gap length, respectively, of DAPAs measured using ImageJ software, and these values are statistical results obtained from 200 DAPAs;

FIG. 5 is a schematic diagram of a localized surface plasmon resonance (LSPR) device according to an embodiment of the present invention;

FIG. 6 is a representative Rayleigh scattering spectrum of a single DAPA during binding of a capture LNA probe, target miRNA, and a detection LNA probe on the surface of the DAPA according to an embodiment of the present invention;

FIG. 7 shows the result of the characterization of the DAPA according to an embodiment of the present invention. Specifically, (A) is an experimentally measured Rayleigh light scattering spectrum of a single DAPA (n=5), wherein the inset is a dark field image of the single DAPA at a magnification of 100×, (B) is a calculated scattering spectrum of the single DAPA computed via finite-difference time-domain (FDTD) simulation, and (C) illustrates the charge density distribution of the single DAPA upon excitation at a specific wavelength that characterizes the longitudinal bonding quadrupole plasmon (LBQP) (top) and the longitudinal bonding dipole plasmon (LBDP) (bottom);

FIG. 8 shows the result of construction of a single DAPA-based label-free plasmonic biosensor according to an embodiment of the present invention. Specifically, (A) is a schematic diagram illustrating the overall experimental procedure for constructing a single DAPA-based plasmonic biosensor for detecting Alzheimer's disease (AD) biomarkers, and (B) shows a representative Rayleigh light scattering spectrum of a single DAPA during binding of a capture LNA probe, target miRNA, and a detection LNA probe on the surface of the DAPA (1: single DAPA fixed on MPTES-treated glass slide, 2: a LNA capture probe-conjugated DAPA, 3: formation of complex of DAPA surface-bound LNA capture probe/target exo-miR/LNA detection probe;

FIG. 9 shows the result of verification of the sensitivity of the single DAPA-based label-free plasmonic biosensor according to an embodiment of the present invention. Specifically, (A) is a graph showing exo-miR-125b detected at a concentration ranging from 10⁰ to 10¹⁰ aM, (B) is a graph showing exo-miR-15a detected at a concentration ranging from 10⁰ to 10¹⁰ aM, (C) is a graph showing exo-miR-361 detected at a concentration ranging from 10⁰ to 10¹⁰ aM, (D) illustrates the linear regression relationship between the LSPR peak shift and exo-miR-125b at a concentration ranging from 10¹ to 10⁹ aM, (E) illustrates the linear regression relationship between the LSPR peak shift and exo-miR-15a at a concentration ranging from 10¹ to 10⁹ aM and (F) illustrates the linear regression relationship between the LSPR peak shift and exo-miR-361 at a concentration ranging from 10¹ to 10⁹ aM. Error bars represent standard deviations from 30 DAPA measurements;

FIG. 10 shows the result of verification of the selectivity of the single DAPA-based label-free plasmonic biosensor using various types of target miRNA according to an embodiment of the present invention. Specifically, (A) shows the result of detection of exo-miR-125b, (B) shows the result of detection of exo-miR-15a and (C) shows the result of detection of exo-miR-361. LSPR spectral shifts were identified using the sequence of target miRNA at a concentration of 10⁹ aM and three types of miRNA sequences including single-point mutations for each target miRNA. Error bars represent standard deviations from 30 DAPA measurements;

FIG. 11 shows the result of verification of the sensitivity of the single DAPA-based label-free plasmonic biosensor according to an embodiment of the present invention. Specifically, (A) shows the result of detection of exo-miR-125b at a concentration ranging from 10⁰ to 10⁷ fM, (B) shows the result of detection of exo-miR-15a at a concentration ranging from 10⁰ to 10⁷ fM and (C) shows the result of detection of exo-miR-361 at a concentration ranging from 10⁰ to 10⁷ fM. The linear regression relationship between the LSPR peak shift and concentrations of (D) exo-miR-125b (E) exo-miR-15a and (F) exo-miR-361 ranges from 10¹ to 10⁶ fM. Error bars represent the standard deviations of 30 DAPA measurements;

FIG. 12 shows the result of selectivity of the single DAPA-based label-free plasmonic biosensor according to an embodiment of the present invention using various types of target miRNA. Specifically, (A) shows the result of detection of exo-miR-125b, (B) shows the result of detection of exo-miR-15a and (C) shows the result of detection of exo-miR-361. LSPR spectral shifts were identified using respective target miRNA and two types of miRNA sequences including single-point mutations for the respective target miRNA. Error bars represent standard deviations from 30 DAPA measurements;

FIG. 13 shows the characterization of exosomes isolated from serum samples according to an embodiment of the present invention. Specifically, (A) is an FE-TEM image of the isolated exosomes, (B) and (C) show the results of NTA and DLS analysis identifying spherical exosomes having a size distribution of 50 to 250 nm, respectively, and (D) shows the result of analysis of total cell lysate (TCL), and ALIX, CD9 and calnexin expression by Western blotting. For reference, the molecular weight of each marker is shown on the right;

FIG. 14 shows the result of clinical diagnosis targeting Alzheimer's disease patients (n=10) and healthy control (HC) (n=6) according to an embodiment of the present invention (***p<0.002), wherein (D) and (E) show receiver operating characteristic (ROC) curves of single exo-miR and a combination of exo-miR, respectively, for differentiating AD patients from the healthy control (HC);

FIG. 15 is box plots showing the estimated qRT-PCR relative expression levels of (A) exo-miR-125b, (B) exo-miR-15a and (C) exo-miR-361 in sera derived from AD patients (n=10) and HC (n=6) according to an embodiment of the present invention (*p<0.05, **p<0.02);

FIG. 16 is a heat map showing the correlation between the miRNA expression level and Alzheimer's disease clinical characteristics (age and MMSE score) according to an embodiment of the present invention (*p<0.05, **p<0.01); and

FIG. 17 shows the receiver operating characteristic (ROC) curves of (A) exo-miR-125b, (B) exo-miR-15a, and (C) exo-miR-361 for differentiating AD patients from the HC according to an embodiment of the present invention.

### DETAILED DESCRIPTION OF THE INVENTION

Hereinafter, the present invention will be described in detail. Meanwhile, each description and embodiment disclosed in the present invention may be applied to other description and embodiment. That is, all combinations of various elements disclosed herein fall within the scope of the present invention. In addition, the following detailed description should not be construed as limiting the scope of the present invention.

In addition, those skilled in the art will recognize or appreciate a number of equivalents to the specific embodiments of the present invention disclosed herein through routine experimentation. Also, such equivalents are intended to fall within the scope of the present invention.

The present invention is devised to overcome the drawbacks and limitations of conventional Western blotting or qRT-PCR in detecting an Alzheimer's disease-related target biomarker.

Accordingly, in one aspect, the present invention is directed to a single metal-DAPA including at least one metal nanosphere conjugated with one single-stranded DNA (1ssDNA) and at least one metal nanosphere conjugated with two single-stranded DNAs (2ssDNA) complementary thereto, the single metal-DAPA having a bridge structure and including a nanogap greater than 0 and not greater than 2 nm.

In another aspect, the present invention is directed to a single DAPA-based label-free nanoplasmonic biosensor.

Specifically, the biosensor includes a substrate, a metal-DAPA (DNA-assembled advanced plasmonic architecture) fixed to the substrate and conjugated with a capture probe specifically binding to a target biomarker, and a measuring device configured to measure localized surface plasmon resonance in the metal-DAPA.

Hereinafter, the present invention will be described in more detail.

As used herein, the term “nanoplasmonic biosensor” refers to a biosensor that is capable of measuring plasmons, wherein the plasmons mean a quantum of an oscillation of electron or hole density, in other words, a quantum of plasma oscillation, and that includes a measuring device for measuring plasmons which are quasiparticles associated with a collection of oscillations of free electrons in a metal.

The biosensor may be a single DAPA-based label-free biosensor.

As used herein, the term “DNA-assembled advanced plasmonic architecture (DAPA)” is an abbreviation for an advanced plasmonic architecture in which DNA is assembled.

The DAPA may include three metal nanospheres conjugated with single-stranded DNA.

In addition, the DAPA may include the 1ssDNA and the 2ssDNA including a sequence complementary thereto at a ratio of 2:1.

The terms “1ssDNA” and “one single-stranded DNA”, which are used interchangeably with each other, refer to DNA including one strand, and the terms “nanosphere conjugated with one single-stranded DNA” and “nanosphere conjugated with 1ssDNA”, which are used interchangeably with each other, refer to a nanosphere in which one single-stranded DNA is fused to one side of the nanosphere.

The terms “2ssDNA” and “two single-stranded DNAs”, which are used interchangeably with each other, refer to two DNA molecules, each including one strand. Specifically, two single-stranded DNAs are linked at an angle of 180 degrees to both sides of a nanosphere. In this case, the 1ssDNA and the 2ssDNA may have complementary sequences to each other to enable hybridization therebetween.

The nanosphere conjugated with 1ssDNA and the nanosphere conjugated with 2ssDNA may be present at a ratio of 2:1, one 1ssDNA may complementarily bind to one strand of the 2ssDNA, and another 1ssDNA may complementarily bind to the remaining strand of the 2ssDNA.

As a result, the three nanospheres can hybridize through complementary binding between the respective molecules of DNA to form a bridge structure.

Any DNA may be used as the DNA without limitation as long as it is capable of complementary binding, and the length of the DNA may be, for example, 75 bp to 150 bp, 85 bp to 120 bp, or 100 bp, but is not limited thereto.

The nanospheres constituting the metal-DAPA may form a bridge structure.

The bridge structure includes a DNA sequence in each of the three nanospheres and is formed by hybridization of the corresponding DNA and then crystallization in the DNA direction at a pH of 5.

The metal-DAPA may include a nanogap.

The nanogap refers to the distance of nanometers in size and may be, for example, greater than 0 and not greater than 2 nm.

The metal may be any one selected from the group consisting of gold (Au), copper (Cu), platinum (Pt), and palladium (Pd), and may be, for example, gold, but is not limited thereto.

The metal-DAPA may be conjugated with a capture probe that specifically binds to a target biomarker.

As used herein, the term “target biomarker” refers to a biomarker to be detected or diagnosed in a form isolated from a biological subject and may be, for example, DNA, miRNA, or peptide, and in another example may be miRNA, but is not limited thereto.

The miRNA is microRNA, which is a short (20-24 nt) non-coding RNA that affects both mRNA stability and translation and is involved in post-transcriptional regulation of gene expression in multicellular organisms. The miRNA may be, for example, derived from exosomes, or may be exosome-derived miRNA obtained from subjects with Alzheimer's disease.

The exosomes are a kind of vesicles (extracellular vesicles, EVs) that are generated within cells and released to the outside of the cells, and are vesicles with a size of 50 to 150 nm that are secreted through information exchange between eukaryotic cells.

The exosome-derived miRNA may be exo-miR125b, exo-miR15a, exo-miR361, or a combination thereof, but is not limited thereto.

As used herein, the term “capture probe” may be used interchangeably with a binding probe or the like, and may be characterized in that it specifically binds to a target or target biomarker to be detected.

The capture probe may include DNA or LNA.

The LNA is used to further improve the sensitivity and selectivity of the sensor, and may include a moiety of a chimeric ribose ring fixed between 2′-oxygen and 4′-carbon through an O-methylene bridge. Such a LNA structure can improve hybridization specificity, duplex stability, and binding affinity of the biosensor.

In a specific embodiment of the present invention, it was found that target exo-miRNA having a complementary sequence completely identical to a probe including two LNAs is bound to the plasmonic biosensor, thereby stably maintaining a sandwich structure and stably providing a detection signal.

The biosensor may detect exosome-derived miRNA proteins by measuring a change in Rayleigh scattering spectrum caused by the specific binding of exosome-derived miRNA.

In addition, the biosensor may detect exosome-derived miRNA in a very small content range of an attomolar level.

According to an embodiment of the present invention, the biosensor may detect exosome-derived miRNA even at a low detection limit, but is not limited thereto.

The term “substrate” refers to a plate to which the metal nanoparticle platform is fixed to allow observation under a microscope and specifically may be a glass slide, but is not limited thereto.

In a specific embodiment of the present invention, a nanoplasmonic biosensor obtained by fixing a produced gold DAPA to a substrate, followed by conjugation with a capture probe including DNA or LNA specific to an isolated target biomarker, for example, exosome-derived miRNA, is capable of effectively detecting even a trace amount of an attomolar level through the detection probe when the target biomarker, exosome-derived miRNA, particularly exo-miR125b, exo-miR15a, exo-miR361, or a combination thereof is injected.

In another aspect, the present invention is directed to a method for detecting exosome-derived miRNA, including treating the single DAPA-based label-free nano-plasmonic biosensor with a biomarker mixture.

The terms “single DAPA-based label-free nanoplasmonic biosensor”, “exosome” and “miRNA” are the same as described above.

As used herein, the term “biomarker mixture” refers to a mixture containing the biomarker to be detected in the present invention. The biomarker mixture may be, for example, plasma, blood, or plasma from which agglutinin has been removed, and in another example, the biomarker mixture may contain exosome-derived miRNA, as another example, may contain exosome-derived miRNA isolated from a subject having Alzheimer's disease, and as another example, may contain exo-miR125b, exo-miR15a, exo-miR361 or a combination thereof. Any mixture may be used as the biomarker mixture without limitation as long as it contains miRNA to be detected in the present invention.

In another aspect, the present invention is directed to a method of diagnosing Alzheimer's disease, including treating the single DAPA-based label-free nano-plasmonic biosensor with a biomarker mixture and treating the single DAPA-based label-free nano-plasmonic biosensor with an exosome-derived miRNA detection probe.

The terms “single DAPA-based label-free nanoplasmonic biosensor”, “biomarker mixture”, “exosome” and “miRNA” are the same as described above.

As used herein, the term “Alzheimer's disease” refers to a disease that is reported to be caused by various complicated pathological and physiological conditions such as amyloid cascades, tau phosphorylation, neurotransmitters, excitotoxicity or oxidative stress, has major pathological features of cerebral accumulation of β-amyloid (Aβ) peptides or neurofibrillary tangles (NFT) of tau, and is closely related to neurodegenerative mechanisms leading to toxicity and destruction of neurons and synapses during pathogenesis thereof.

As used herein, the term “detection probe” refers to a probe for detecting target miRNA that complementarily binds to a capture probe bound to the nanosphere having a bridge structure included in the biosensor, for example, a probe that complementarily binds to target miRNA to detect the target miRNA. In another example, the probe may include DNA or LNA, but is not limited thereto.

In another aspect, the present invention is directed to a method of preparing a single DAPA-based nanoplasmonic biosensor, the method including (a) hybridizing one single-stranded DNA (1ssDNA)-metal nanoparticles with two single-stranded DNAs (2ssDNA)-metal nanoparticles having a sequence complementary thereto at a ratio of 2:1 to obtain metal-DAPA seeds, (b) coating the metal-DAPA seeds with a predetermined material, (c) crystallizing the coated metal-DAPA seeds by treatment with a metal precursor and a reducing agent, (d) fixing the metal-DAPA to a substrate and (e) conjugating the metal-DAPA with a capture probe specifically binding to an isolated target biomarker.

The terms “single DAPA-based label-free nanoplasmonic biosensor”, “one single-stranded DNA (1ssDNA)-metal nanoparticle”, “two single-stranded DNAs (2ssDNA)-metal nanoparticle having a sequence complementary thereto”, “hybridization”, “metal-DAPA”, “capture probe”, and “substrate” are the same as described above.

As used herein, the term “coating” refers to a process of covering the outer surface of an object with a specific material to form a thin film and the coating may be performed with, for example, polyethylene glycol (PEG), but is not limited thereto.

As used herein, the term “crystallization” refers to a process of crystallizing the metal-DAPA by treating the metal-DAPA with a metal precursor and a reducing agent to reduce AuCl₄⁻ to Au atoms during nanostructure synthesis, and metal crystallization may occur along the double stranded DNA (dsDNA) than the surface of the metal NP.

In addition, the crystallization may be performed at a pH adjusted to an appropriate range, for example, at a pH of 5, so that the bridge structure can be better formed.

The method may further include coating the substrate with (3-mercaptopropyl)trimethoxysilane (MPTES) before step (d).

In another aspect, the present invention is directed to a method of preparing a single metal-DAPA, the method including (a) hybridizing one single-stranded DNA (1ssDNA)-metal nanoparticles with two single-stranded DNAs (2ssDNA)-metal nanoparticles having a sequence complementary thereto at a ratio of 2:1 to obtain metal-DAPA seeds, (b) coating the metal-DAPA seeds with a predetermined material and (c) crystallizing the coated metal-DAPA seeds by treatment with a metal precursor and a reducing agent.

The terms “single DAPA-based label-free nanoplasmonic biosensor”, “one single-stranded DNA (1ssDNA)-metal nanoparticle”, “two single-stranded DNAs (2ssDNA)-metal nanoparticle having a sequence complementary thereto”, “hybridization”, “metal-DAPA”, “coating”, and “crystallization” are the same as described above.

In another aspect, the present invention is directed to the use of a single metal-DAPA including metal nanospheres conjugated with one single-stranded DNA (1ssDNA) and metal nanospheres conjugated with two single-stranded DNAs (2ssDNA) complementary thereto, the single metal-DAPA including a nanogap greater than 0 and not greater than 2 nm.

The terms “metal nanosphere conjugated with one single-stranded DNA (1ssDNA),” “metal nanosphere conjugated with two single-stranded DNAs (2ssDNA) complementary thereto,” “nanogap” and “single metal-DAPA” are the same as described above.

Hereinafter, the present invention will be described in more detail with reference to specific examples. These examples are merely provided for illustration of the present invention, and it will be apparent to those skilled in the art that these examples should not be construed as limiting the scope of the present invention. Accordingly, the substantial scope of the present invention will be defined by the appended claims and equivalents thereto.

### EXPERIMENTAL EXAMPLE 1

### Materials

Anhydrous ethyl alcohol (CH₃CH₂OH, ≥99.5%), a bis (p-sulfonatophenyl)phenylphosphine dihydrate dipotassium salt (BSPP), 1,4-dithiothreitol (DTT), ethyl acetate (anhydrous, 99.8%), ethanolamine, gold (III) chloride trihydrate (99.0%), hydrochloric acid (HCl, 37 wt % in water), 3-mercaptopropyl)trimethoxysilane (MPTES), sodium dodecyl sulfate (SDS), and sodium chloride (NaCl) were obtained from Sigma-Aldrich Korea Ltd. (Korea). Methoxypolyethylene glycol-thiol (mPEG-SH) was obtained from Futurechem (Korea).

A gold nanoseed (AuNS; 15 nm) solution was obtained from British BioCell International (UK). Saline sodium citrate (SSC) buffer, 5× TBE buffer, and diethyl pyrocarbonate (DEPC)-treated water were obtained from Biosesang Co., Ltd. (Korea). Coverslip slides (22×40×0.1 mm) were obtained from Deckglaser (Germany), and DNA LoBind tubes were obtained from Eppendorf (Germany). Capture/detection DNA probes, target miRNA, and single-point-mutated miRNA were obtained from Bioneer (Korea). The total exosome RNA isolation kit was obtained from Thermo Fisher Scientific (USA). Healthy control sera and AD patient sera were obtained from PrecisionMed, Inc. (USA). Ultrapure water (18.2 mΩ cm⁻¹) was used to prepare all solutions.

### EXPERIMENTAL EXAMPLE 2

### Preparation of Gold Nanoparticles (AuNP) Conjugated with Single-Stranded DNA (ssDNA)

100 mL of a solution of AuNPs (15 nm) was mixed with 330 mg of a bis(p-sulfonatophenyl)phenylphosphine dihydrate dipotassium salt (BSPP), followed by incubation overnight. Then, the resulting solution was mixed with 15.0 g of NaCl and centrifuged at 500×g for 30 minutes, the supernatant was removed, and the precipitate was resuspended in 1 mL of a BSPP (0.5 mM) solution and 0.5 mL of MeOH. AuNPs were centrifuged again (500×g, 30 min) and then dissolved in 1 mL of 0.5×TBE buffer containing 0.5 mM BSPP. The optical density of the collected AuNPs was measured using an ultraviolet-visible-near-infrared spectrophotometer (UV-3600; Shimadzu, Kyoto, Japan). 100-bp 5′ thiol-modified nucleotides were treated with DTT solution for 15 minutes and then separated three times using ethyl acetate. Then, the phosphine-stabilized AuNPs were incubated overnight along with ssDNA at a molar ratio of 1:2.5 in 0.5× TBE buffer containing 40 mM NaCl at 25° C. Then, the ssDNA-conjugated AuNPs were separated through gel electrophoresis on a 3.0% agarose gel at 75V for 1 hour. Bands including AuNP-1DNA and AuNP-2DNA were isolated and eluted in 0.5×TBE buffer for several days. The eluted product was concentrated at 10,000 rpm for 30 minutes and absorbance was measured at 450 nm to calculate the concentration of nanoparticles.

### EXPERIMENTAL EXAMPLE 3

### Isolation of Exosomal miRNA from Clinical Samples

Serum samples were loaded and eluted using PBS buffer in accordance with the protocol. The isolated exosomes were concentrated to a final volume of 200 μL using an Amicon Ultra 0.5 mL centrifuge filter (Merck Millipore), and the morphology of exosomes was observed using a JEM-1400 Plus at 120 kV and a JEM-1000BEF at 1000 kV (JEOL Ltd., Japan). Serum-derived exosomal miRNA was separated using a total exosomal RNA and protein isolation kit (Invitrogen, USA) in accordance with the manual therefor, and the concentration of exosomal miRNA was measured with a nanodrop spectrophotometer (NANODROP2000, ThermoFisher science, USA).

### EXPERIMENTAL EXAMPLE 4

### Quantitative Reverse Transcription Polymerase Chain Reaction (qRT-PCR) Analysis

The content of exosomal miRNA derived from serum of six healthy control (HC) subjects and ten AD patients was estimated using qRT-PCR. Total RNA (2 ng/μL) was subjected to reverse transcription into cDNA using a TaqMan miRNA reverse transcription Kit (ThermoFisher Scientific, USA) at 16° C. for 30 minutes, at 42° C. for 30 minutes, at 85° C. for 5 minutes, and at 4° C. for 5 minutes. 42 cycles of real-time qPCR was performed using a TaqMan miRNA assay kit (ThermoFisher science, USA) and an Applied Biosystems StepOnePlus real-time PCR system (Applied Biosystems) at 95° C. for 10 minutes, at 95° C. for 15 seconds, and then at 60° C. for 1 minute. The cycle threshold (Ct) was created using the SDS program (v2.0.1; Applied Biosystems) and the relative expression level of the target exosomal miRNA was calculated using the internal normalization control (U6) after normalization.

### EXPERIMENTAL EXAMPLE 5

### Statistical Analysis

The correlation between AD-related clinical parameters (age and MMSE) and miRNA expression levels was investigated based on Pearson correlation using OriginPro 8 software (OriginLab). Whether the correlation was statistically significant was determined using the standard Student's two-tailed t-test. p<0.05 was considered statistically significant.

### EXAMPLE 1

### DAPA Design Using Finite-Difference Time-Domain (FDTD) Simulation

In order to calculate the scattering spectrum and electric field (EF), FDTD simulation was performed using the Lumerical FDTD solution program (Lumerical Inc., Vancouver, BC, Canada).

AuNP is effectively capable of converting the binding between the biomolecular analyte and the receptor fixed on the NP surface into physical and electrochemical signals in a single NP (nanoparticle)-based plasmonic biosensor. Structural and geometrical parameters of NPs, such as the size, shape and aspect ratio thereof, greatly affect the spectral sensitivity of plasmonic biosensors. AuNRs have higher RI sensitivity than spherical NPs because the longitudinal plasmons absorb more light, thus enhancing EF. Therefore, the DAPA was designed based on the rod-shaped structure.

Specifically, three gold nanospheres (AuNS, each having a diameter of 15.8 nm) were connected using a gold (Au) plate (having a diameter of 11.2 nm) to produce a DAPA having a final aspect ratio (AR) of 3.04.

Then, in order to significantly improve the spectral sensitivity of the plasmonic structure, two narrow nanogaps (2 nm) were formed in the rod-shaped structure.

In order to explain the plasmon-coupling effect of the nanogap on the produced DAPA of the present invention and electric field (EF) distribution, nanospheres having gaps therebetween, gold nanorods having gaps therebetween (AuNR; AR 3.04), three nanospheres having no bridge structure, and nanoprobes having a bridge structure were produced, and the EF distribution of the DAPA of the present invention was compared with that of each of the other Au nanostructures. The wavelength range of the incident light was set to 400 to 1,000 nm, the mesh size was set to 0.5 nm, and the sensitivity was determined by changing the RI of the surrounding medium from 1.333 to 1.45.

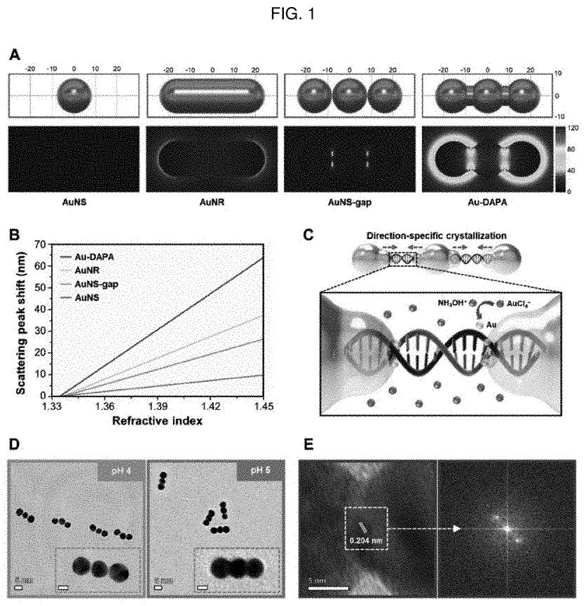

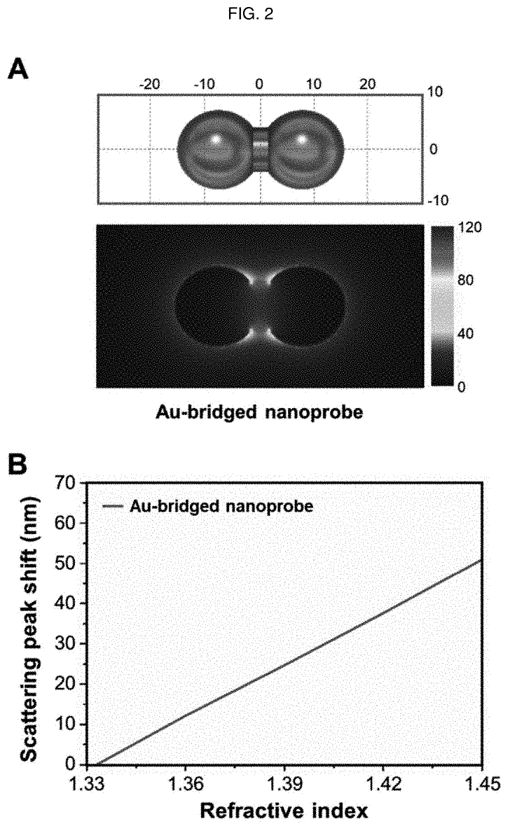

As can be seen from FIGS. 1 and 2, the Au-DAPA of the present invention forms a nanogap, which allows the EF distribution to be strong over the entire surface of Au-DAPA due to compression of incident light based thereon, and creation of a hotspot that amplifies electromagnetic energy tens of times. In particular, it can be seen that a nanogap of 2 nm or less has strong potential for analysis of small-sized biomolecules such as DNA, miRNA or peptides, and even a small change in the nanogap may greatly increase a plasmon resonance shift due to the localized optical field of the nanogap.

Then, the RI sensitivity of the Au-DAPA of the present invention was evaluated by simulating the scattering peak shift to the surrounding RI change (FIG. 1B). The RI sensitivity of the scattering peak is reported to be directly relevant to the detection sensitivity of the plasmonic biosensor, since single NP-based plasmonic biosensors can detect target biomarkers through the scattering peak shift in response to the local change in RI.

As a result, as can be seen from FIG. 1B, the RI sensitivity of Au-DAPA was increased 1.66 times compared to that of a conventional nanorod structure known to exhibit strong RI sensitivity, and, as can be seen from FIG. 2B, the Au-DAPA structure of the present invention, in which three nanospheres were bridged, had stronger RI sensitivity than a structure in which two nanospheres were bridged.

This indicates that the Au-DAPA structure according to the present invention, produced by hybridizing 1ssDNA-AuNP and 2ssDNA-AuNP having a sequence complementary thereto at a ratio of 2:1, has the strongest RI sensitivity and is thus more effectively used for a nanoplasmonic biosensor.

### EXAMPLE 2

### DAPA (DNA-Assembled Advanced Plasmonic Architecture) Synthesis

One single-stranded DNA-gold nanoparticles (1ssDNA-AuNP) and two single-stranded DNAs-gold nanoparticles (2ssDNA-AuNP) having a sequence complementary thereto were hybridized at a ratio of 2:1 and incubated at 37° C. for 3 hours to obtain seeds of DAPA. The seeds were purified using gel electrophoresis on a 2.0% agarose gel at 100V for 20 minutes. The band including the seeds was immersed in 0.5× TBE buffer containing 100 mM NaCl, and the seed surface was coated with neutral polyethylene glycol (PEG) at a molar ratio of 1:100 at 25° C. for 16 hours. Then, the seeds (0.5 nM) were gently stirred along with 4.5 μL of a gold precursor and 7.90 μL of a reducing agent for 1 hour and 3 hours, respectively. After the gold precursor (HAuCl₄, 0.03%) and reducing agent (NH₂OH·HCl, 1 mM) solution were adjusted to pH 4.0 and 5.0, respectively, the obtained final product was washed at 8,000 rpm for 30 minutes and resuspended in ultrapure water.

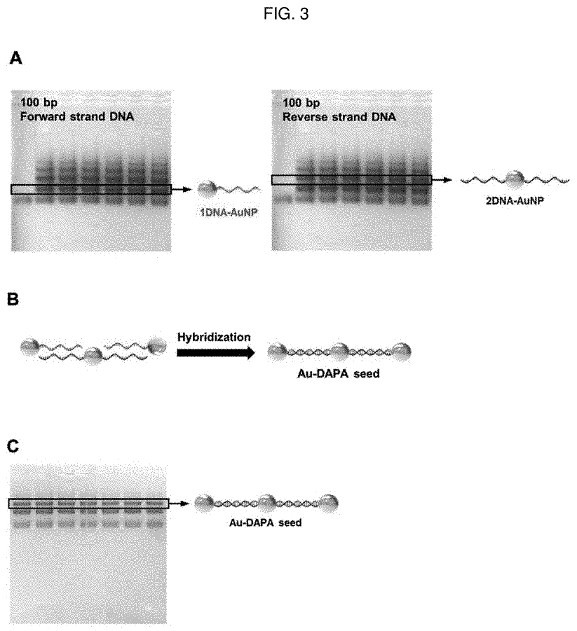

Then, whether or not the pre-designed and synthesized DAPA actually had improved optical properties was determined through FDTD simulation. The seeds of DAPA were prepared through hybridization between two types of ssDNA-AuNPs (1ssDNA-AuNP and 2ssDNA-AuNP), in which 100 bp single-stranded DNA (ssDNA) was conjugated with 15 nm spherical AuNP (FIG. 3), and then the AuNP surface was modified with PEG. After hybridization, the double-stranded DNA (dsDNA) of the seeds provides a direction-specific guide, and the PEG-coated AuNPs serve to lower the activation energy required to reduce AuCl₄⁻ to Au atoms during the synthesis of colloidal nanostructures. Therefore, Au crystallization occurs more rapidly along the dsDNA strand than on the surface of AuNPs. In addition, the DAPA synthesis pattern depending on pH was determined, since the surface charge of DNA is affected by pH. As a result, as can be seen from FIG. 1C, the isoelectric point (pI) of dsDNA ranges between 4.0 and 4.5 at a pH of 5, so double-stranded DNA (dsDNA) was slightly negatively charged. As a result, AuCl₄⁻ ions reacted with high-concentration NH₃OH⁺ ions surrounding the dsDNA to initiate Au crystallization at the AuNP/DNA interface and allow the Au crystals to grow along the dsDNA contour (FIG. 1C). On the other hand, at a pH of 4, crystallization was not complete, so a gap of 0.96 nm remained between AuNS.

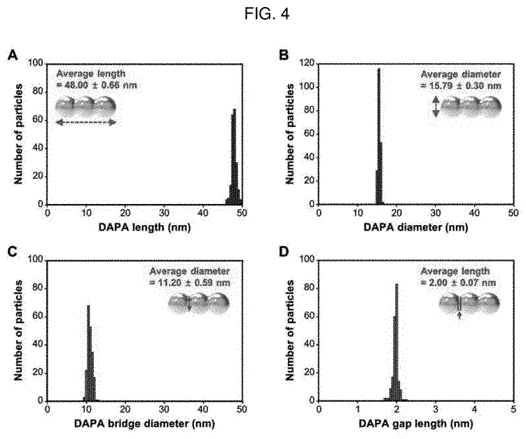

Eventually, this method showed that the Au-DAPA of the present invention, having a length of 48.00±0.66 nm, a diameter of 15.79±0.30 nm, a bridge diameter of 11.20±0.59 nm, and a gap of 2.00±0.07 nm, was uniformly synthesized (FIGS. 1D and 4).

It was confirmed that the DNA-directed region, that is, the bridge, had a crystal lattice plane spacing of 0.204±0.005 nm according to the lattice pattern in the growth direction of the face-centered cubic (FCC) crystal structure of Au (FIG. 1E).

Then, for characterization of DAPA, 10 μL of the sample was dropped onto a 400 mesh copper grid (F/C, Ted Pella, Inc., Redding, Calif., USA) and dried overnight at 25° C. Energy filtering TEM (EF-TEM; LIBra 200 FE, Carl Zeiss AG, Germany) was used to obtain images of DAPA. The morphology of DAPA, including the length, diameter, bridge diameter, and gap length, was analyzed using ImageJ software. The crystal structure of DAPA was confirmed using field emission TEM (FE-TEM; Tecnai G2 F30ST, FEI company, USA).

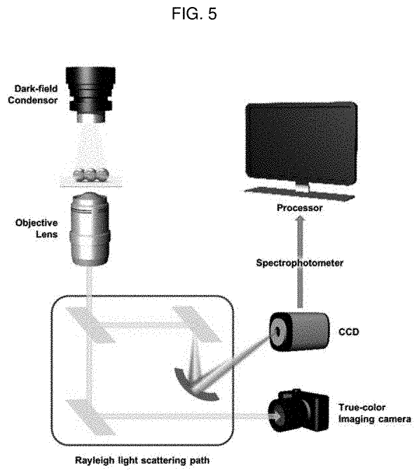

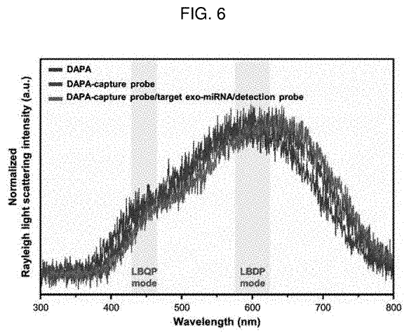

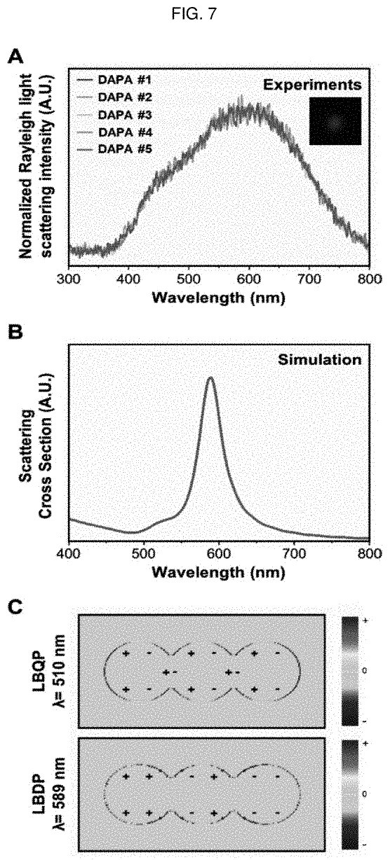

First, in order to determine the optical properties of DAPA, the scattering spectrum of a single DAPA was measured using an experimental apparatus (FIGS. 5 and 6). As a result, as can be seen from FIG. 6, the scattering spectrum of a single DAPA showed two unique peaks near 590 nm and 460 nm, similar to the nanorod structure having the same AR (FIG. 6). In particular, the DAPA having a narrow nanogap structure exhibited a distinct intensity of the scattering spectrum due to the plasmon-coupling effect. In addition, it was found that the size and shape of the synthesized particles were essentially very homogeneous based on the consistency of the LSPR peak positions of individual DAPAs formed at 589.81±0.75 nm (FIG. 7A).

Then, the scattering spectrum of the single DAPA was further calculated using FDTD simulations to determine whether or not the experimentally measured spectra were accurate. As a result, as can be seen from FIG. 7B, the calculated Rayleigh scattering spectrum corresponded closely to the experimentally measured spectrum (FIG. 7B). The calculated charge density of DAPA showed that the longer wavelength region (589 nm) was assigned to the longitudinal bonding dipole plasmon (LBDP) mode, whereas the 510 nm peak was attributed to the longitudinal bonding quadrupole plasmon (LBQP) mode (FIG. 7C). However, the quadrupole excitation response was not as clearly observed as the dipolar band because it is a sub-radiant mode that allows weak bonding to incident light. Based on these results, it can be seen that the DAPA of the present invention is applicable as a sensing material due to the uniformity and signal reproduction capability thereof.

### EXAMPLE 3

### Production of Single DAPA-Based Label-Free Plasmonic Biosensor

In order to detect AD biomarkers based on a single DAPA, a label-free plasmonic biosensor, that is, a plasmonic biosensor having no label, was produced.

Specifically, to produce a DAPA-based label-free plasmonic biosensor, 10 μL of a DAPA solution (OD˜0.01) was dropped onto an MPTES-treated microscope coverslip slide and dried at 25° C. for 2 hours. A DAPA-fixed coverslip slide was then inserted into an imaging chamber (RC-30, Warner Instruments, Hamden, Conn., USA), inserted into a dark field microscope holder (Marzhauser Sensotech, Wetzlar, Germany), and then fixed on a syringe pump (PHD2000, Harvard Apparatus, Holliston, Mass., USA). Then, in order to remove unfixed DAPA and contaminants, ultra-pure water was injected at 160 μL/min into the chamber for 1 hour. To modify the surface of DAPA with a 22-bp (base pair) DNA or LNA capture probe, the capture probe (1 μM) was incubated in 3×SSC/0.04% SDS at 30° C. for 6 hours, and unbound capture probes were removed with a wash solution (2×SSC/0.1% SDS) at 160 μL/min for 5 minutes. In order to inhibit non-specific binding, the non-reactive surface of DAPA was treated with 0.1 M ethanolamine for 30 minutes and washed with a wash solution. Then, exo-miR contained in 5×SSC/DEPC-treated water was injected into capture-probe-conjugated DAPA and incubated in an incubator (FinePCR, Seoul, Korea) at 42° C. for 16 hours. Unhybridized exo-miR (exo-miR) was removed at 42° C. using a preheated wash solution. Finally, the detection probe (1 μM) was incubated in 5×SSC/0.1% SDS/DEPC-treated buffer at 60° C. for 4 hours, and excess detection probe was incubated with 2×SSC/0.1% SDS/DEPC-treated buffer for 5 minutes. After each incubation step, the change in the distinct Rayleigh scattering spectrum of a single DAPA was measured at −70° C. under a 100 W halogen source (Type 7724, Philips) using a spectrophotometer (Microspec 2300i, Roper Scientific, France) and a charge-coupled device (CCD) camera (PIXIS: 400B, Princeton Instruments, USA) (Song et al., 2020). The LSPR spectral change was calculated in accordance with the Lorentzian algorithm (λmax (after reaction)−λ max (before reaction) using OriginPro 8 software (OriginLab, Northampton, USA).

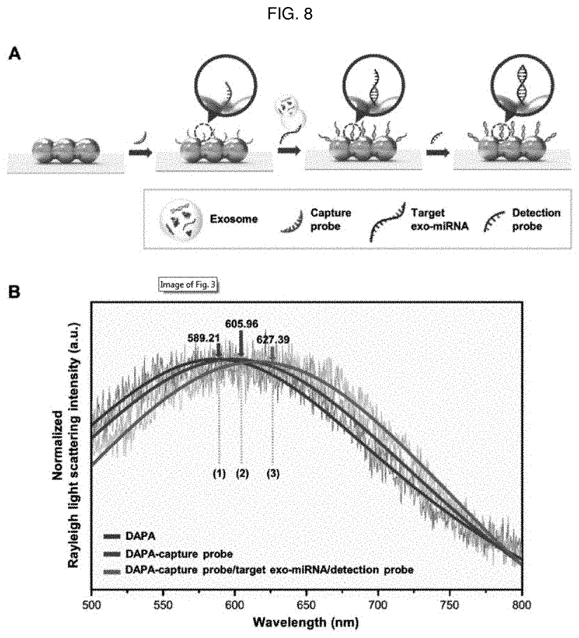

Meanwhile, blood-derived exo-miR-125b, exo-miR-15a, and exo-miR-361, which are currently attracting attention as diagnostic biomarkers for diagnosing Alzheimer's disease, were used as Alzheimer's model analytes. Upregulation of blood-derived exo-miR-125b, exo-miR-15a, and exo-miR-361 in the sera of Alzheimer's disease patients is strongly associated with Alzheimer's disease pathogenesis, such as tau phosphorylation and promoted apoptosis. Therefore, the plasmonic biosensor of the present invention was designed to detect a target miRNA by forming a capture probe/target exo-miR/detection probe complex on the DAPA surface based on the principle of sandwich analysis (FIG. 8A).

In addition, an LNA probe was introduced to further improve the sensitivity and selectivity of the sensor. The structure of LNA containing a moiety of a chimeric ribose ring fixed between 2′-oxygen and 4′-carbon via an O-methylene bridge greatly contributes to the improvement in the hybridization specificity, duplex stability, and binding affinity of the biosensor.

Therefore, the plasmonic biosensor of the present invention binds to target miRNA having a complementary sequence completely identical to the two probes, thereby stably maintaining the sandwich structure and stably providing a detection signal. Detailed information on the sequences of target miRNA and capture/detection probe used in the present invention is shown in Table 1 below.

TABLE 1
Information on nucleotide sequences of target miRNA, LNA
capture/detection probe, and DNA capture/detection probe

| | | | Length | Tₘ |
|---|---|---|---|---|
| Assigned name | | Sequence | (bp) | (° C.) |
| miRNA | miR-125b | SEQ ID NO: 1 | 22 | |
| | miR-125b A | SEQ ID NO: 2 | 22 | |
| | miR-125b B | SEQ ID NO: 3 | 22 | |
| | miR-125b C | SEQ ID NO: 4 | 22 | |
| | miR-15a | SEQ ID NO: 5 | 22 | |
| | miR-15a A | SEQ ID NO: 6 | 22 | |
| | miR-15a B | SEQ ID NO: 7 | 22 | |
| | miR-15a C | SEQ ID NO: 8 | 22 | |
| | miR-361 | SEQ ID NO: 9 | 22 | |
| | miR-361 A | SEQ ID NO: 10 | 22 | |
| | miR-261 B | SEQ ID NO: 11 | 22 | |
| | miR-361 C | SEQ ID NO: 12 | 22 | |
| LNA | miR-125b | SEQ ID NO: 13; 5′ end of | 11 | 71.0 |
| capture | | SEQ ID NO: 13 is thiolated | | |
| probe | | and residue nos. 2, 4, 6, 7, | | |
| | | and 10 are substituted | | |
| | | with LNA | | |
| | miR-15a | SEQ ID NO: 14; 5′ end of | 11 | 73.0 |
| | | SEQ ID NO: 14 is thiolated | | |
| | | and residue nos. 2, 3, 7, | | |
| | | and 8 are substituted | | |
| | | with LNA | | |
| | miR-361 | SEQ ID NO: 15; 5′ end of | 11 | 72.0 |
| | | SEQ ID NO: 15 is thiolated | | |
| | | and residue no. 5 are | | |
| | | substituted with LNA | | |
| LNA | miR-125b | SEQ ID NO: 16; residue nos. | 11 | 71.0 |
| detection | | 4 and 9 are substituted | | |
| probe | | with LNA | | |
| | miR-15a | SEQ ID NO: 17; residue nos. | 11 | 72.0 |
| | | 3, 6, and 8 are substituted | | |
| | | with LNA | | |
| | miR-361 | SEQ ID NO: 18; residue nos. | 11 | 71.0 |
| | | 4, 5, 7, and 9 are | | |
| | | substituted with LNA | | |
| DNA | miR-125b | SEQ ID NO: 19; 5′ end of | 11 | |
| capture | | SEQ ID NO: 19 is thiolated | | |
| probe | miR-15a | SEQ ID NO: 20; 5′ end of | 11 | |
| | | SEQ ID NO: 20 is thiolated | | |
| | miR-361 | SEQ ID NO: 21; 5′ end of | 11 | |
| | | SEQ ID NO: 21 is thiolated | | |
| DNA | miR-125b | SEQ ID NO: 22; GGTCTCAGGGA | 11 | |
| detection | miR-15a | SEQ ID NO: 23; ATGTGCTGCTA | 11 | |
| probe | miR-361 | SEQ ID NO: 24; GATTCTGATAA | 11 | |

In addition, in order to confirm the effectiveness of this platform, the LBDP peak of a single DAPA was analyzed to monitor the hybridization, since the LBDP mode was more sensitive to changes in the dielectric constant of the surrounding medium of the DAPA than the LBQP mode (FIG. 6).

As a result, as can be seen from FIG. 8A, the capture probe having the 5′ end modified with a thiol group (—S) was covalently bound (Au—S) to the surface of DAPA fixed on a coverslip slide (steps 1-2 in FIG. 8B). An LSPR redshift λ max of 16.53±0.76 nm generated on the platform indicates that the capture probe was successfully bound to the DAPA surface. Then, specific hybridization was induced, and the target miRNA and the detection probe were sequentially added to the DAPA surface. The probe was bound to the target miRNA through hydrogen bonding and n-n stacking to generate a saturated LSPR redshift λ max of 21.49±0.95 nm (steps 2-3 in FIG. 8B). These results indicate that the capture/detection probe was perfectly hybridized with the target exo-miR in the serum and formed a stable sandwich structure on a single DAPA surface.

This indicates that the plasmonic biosensor of the present invention can effectively detect miRNA present in a complicated sample without a labeling process.

### EXAMPLE 4

### Verification of Performance of Single DAPA-Based Label-Free Plasmonic Biosensor

In general, the qualitative and quantitative analysis processes of biosensors are greatly affected by various parameters such as analyte concentration, instrument sensitivity, detection limit, linearity and selectivity. Among them, the limit of detection (LOD), which is an essential aspect of sensor performance, should be considered because it represents the minimum concentration of an analyte that is capable of reliably detecting a target molecule. In particular, biosensors that need to detect biomarkers present at extremely low concentrations in human biological samples should have a lower LOD and a wider dynamic range than target biomolecules.

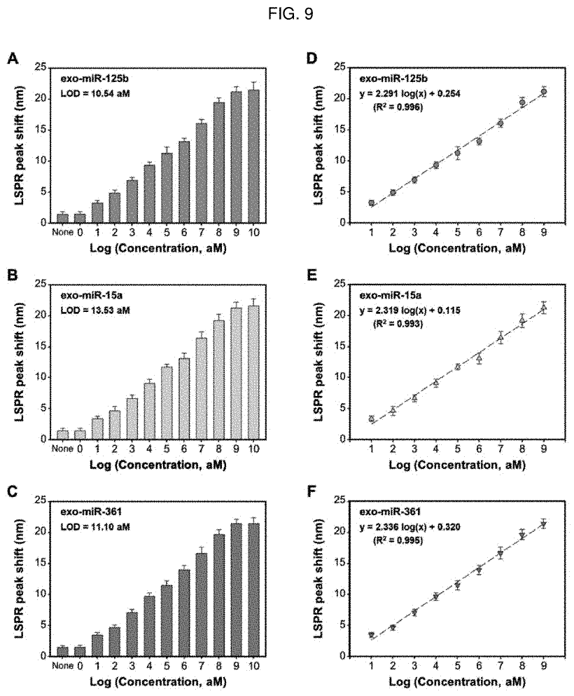

In this regard, in order to quantitatively determine the sensitivity of the plasmonic biosensor of the present invention, the LSPR signal change was measured by changing the concentrations of three model analytes, exo-miR-125b, exo-miR-15a, and exo-miR-361, in the mimicked serum (FIGS. 9A-C). It was found that, although the respective target exo-miRs had different orders, they had a strong linear relationship between the LSPR redshift λ max and logarithmic concentration within the range of 10¹ to 10⁹ aM (FIGS. 9D-F).

Here, the linear regression equation was calculated as follows:

for exo-miR-125b, y=2.291 log(x)+0.254 (R2=0.996) (1)

for exo-miR-15a, y=2.319 log(x)+0.115 (R2=0.993) (2)

for exo-miR-361, y=2.336 log(x)+0.320 (R2=0.995), (3)

wherein x is the concentration of each exo-miR, and the coefficient of determination (R2) is greater than 0.99.

Based on this equation, the LODs of the plasmonic sensors for AD biomarkers were calculated as 10.54, 13.53, and 11.10 aM for exo-miR-125b, exo-miR-15a, and exo-miR-361, respectively. These values were 10³-10⁴ times lower than the miRNA expression levels in the blood of Alzheimer's disease patients and the previously reported miRNA-detecting LSPR sensor (Table 2).

TABLE 2
Comparison in sensing performance between DNA-assembled advanced
plasmon architecture (DAPA)-based plasmonic biosensor according
to present invention and other conventionally known sensors

| | | | | Dynamic | Sample | |
|---|---|---|---|---|---|---|
| Method | Target | LOD | | range | type | Disease |
| LSPR | miR-125b | 10.54 | aM | 10 aM-1 aM | Serum | AD |
| | miR-15a | 13.53 | aM | | | |
| | miR-361 | 11.10 | aM | | | |
| LSPR | miR-10-b | 2.45 | pM | 5 pM-10 pM | DSN | Gastric |
| | | | | | | Cancer |
| LSPR | miR-let-7a | 13.00 | fM | nM-50 nM | PBS | Gastric |
| | | | | | | Cancer |
| LSPR | miR-205 | 5.00 | pM | 0 pM-1 μM | Serum | Lung |
| | | | | | | Cancer |
| LSPR | miR-27a | 16.50 | fM | 00 fM-3 pM | PBS | Breast |
| | | | | | | Cancer |
| EIS | miR-137 | 1.70 | fM | fM-750 fM | Serum | AD |
| EIS | miR-34a | 261.7 | nM | nM-1.45 μM | PBS | AD |
| FRET | miR-29a | 745 | pM | nM-20 nM | Serum | AD |
| Fluorescence | miR-137 | 82 | pM | 0.05 nM-5 nM | Serum | AD |
| and GO | miR-142 | | | | | |

* LSPR: Localized Surface Plasmon Resonance, EIS: Electrochemical Impedance Spectroscopy, FRET: Fluorescence Resonance Energy Transfer, GO: Graphene Oxide, DSN: Duplex-specific nuclease

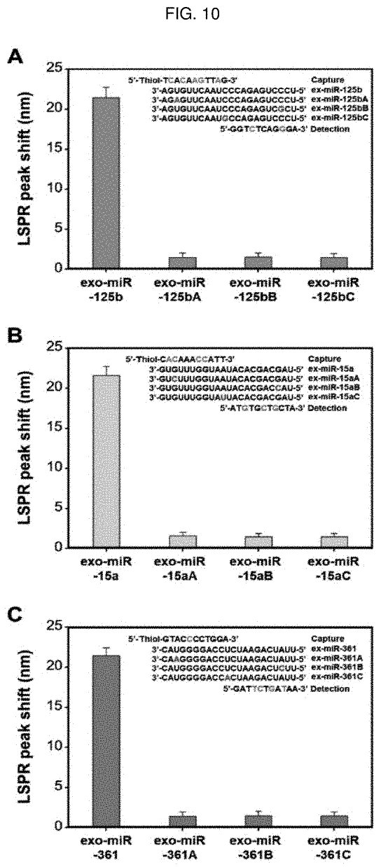

More than 17,000 mature miRNAs have been identified in the human blood, and there is a difference in only one or two nucleotides between familial groups. Therefore, sensors that analyze miRNAs must be capable of discriminating even single-nucleotide differences between these highly homologous miRNAs. In this regard, to investigate the selectivity of the biosensor of the present invention, three additional miRNAs respectively having single-point mutations at the 3′-end (type A), the 5′-end (type B), and the middle (type C) for each miRNA were prepared. Among the three target exo-miR sequences (Table 1), the signals generated on the sensor surface were analyzed. As a result, the target exo-miR having a sequence perfectly complementary to the sequence of the capture and detection probes exhibited an LSPR signal shift of about 21.00 nm on the sensor surface, whereas the sequence of the prepared exo-miR having a point mutation was not consistent. The binding sites of the capture and detection probes generated a slight LSPR redshift λ max of less than 2.00 nm (FIG. 10). These results suggest that the single DAPA-based plasmonic biosensor of the present invention can accurately detect the target exo-miR at an attomolar level without being disturbed by interfering substances in the blood.

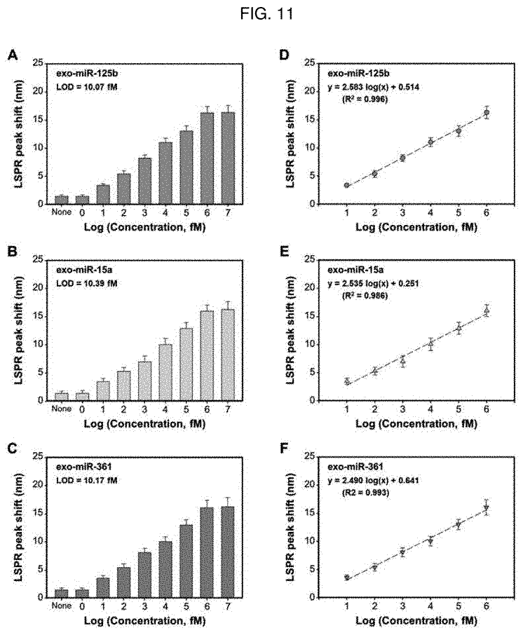

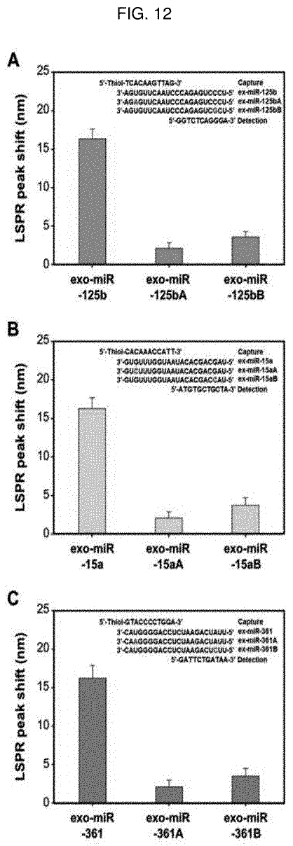

It is considered that this is because i) the 2.0 nm narrow gap of DAPA improves the EF of the nanostructured surface and increases the RI sensitivity, and ii) the introduction of the LNA probe greatly increases the affinity and selectivity of DAPA compared to the conventional DNA probe. The LOD of the biosensor of the present invention was found to be at least 768 times lower than the LOD of the biosensor to which the conventional DNA probe is applied (FIG. 11) and to accurately identify the target miRNA compared to the DNA probe (FIG. 12). These results suggest that the plasmonic biosensor of the present invention is clinically applicable to the diagnosis of Alzheimer's disease.

### EXAMPLE 5

### Application to Clinical Diagnosis

Exo-miR directly reflects the physiological state of the brain, and is highly expressed in cerebral and bodily fluids (for example, CSF, serum, plasma, and urine). Therefore, exosome blood-derived miRNA has great potential usefulness as a biomarker in the diagnosis, prognosis and treatment of Alzheimer's disease. To evaluate the clinical applicability of the sensor, the expression levels of three target miRNAs in exosomes isolated from ten Alzheimer's disease patients and six healthy control (HC) subjects were observed. Detailed information on the AD patients and HCs is shown in Table 3 below.

TABLE 3
Information of Alzheimer's disease (AD)
patients and healthy control (HC) subjects

| Patients | | | | | MMSE | | | |
|---|---|---|---|---|---|---|---|---|
| number | Type | Gender | Ethnicity | Age | score | ADAS | CDR | Diagnosis |
| 8148 | Serum | M | Caucasian | 80 | 19 | 52 | 2 | AD |
| 8238 | Serum | M | Caucasian | 80 | 15 | 52 | 1 | AD |
| 8327 | Serum | F | Caucasian | 71 | 19 | 32 | 0.5 | AD |
| 8339 | Serum | F | Caucasian | 72 | 14 | 53 | 2 | AD |
| 8377 | Serum | M | Caucasian | 79 | 17 | 31 | 1 | AD |
| 8420 | Serum | F | Caucasian | 73 | 19 | 27 | 1 | AD |
| 8464 | Serum | F | Caucasian | 76 | 16 | 32 | 1 | AD |
| 8471 | Serum | M | Caucasian | 76 | 20 | 30 | 1 | AD |
| 8566 | Serum | F | Caucasian | 74 | 16 | 43 | 1 | AD |
| 8567 | Serum | F | Caucasian | 69 | 21 | 33 | 1 | AD |
| 8201 | Serum | F | Caucasian | 73 | 30 | | | HC |
| 8204 | Serum | M | Caucasian | 73 | 30 | | | HC |
| 8210 | Serum | M | Caucasian | 65 | 30 | | | HC |
| 8212 | Serum | F | Caucasian | 70 | 30 | | | HC |
| 8213 | Serum | M | Caucasian | 73 | 30 | | | HC |
| 8218 | Serum | F | Caucasian | 67 | 30 | | | HC |

MMSE: Mini-Mental State Examination; maximum score is 30 points (20-24: mild dementia, 13-20: moderate dementia, <12: severe dementia). ADAS: Alzheimer's disease assessment scale; maximum score is 70 points (higher scores (≥18) indicate greater severity of cognitive impairment). CDR: clinical dementia grade; maximum score is 3 (0: no dementia, 0.5: suspected dementia, 1: mild cognitive impairment, 2: moderate cognitive impairment, 3: severe cognitive impairment).

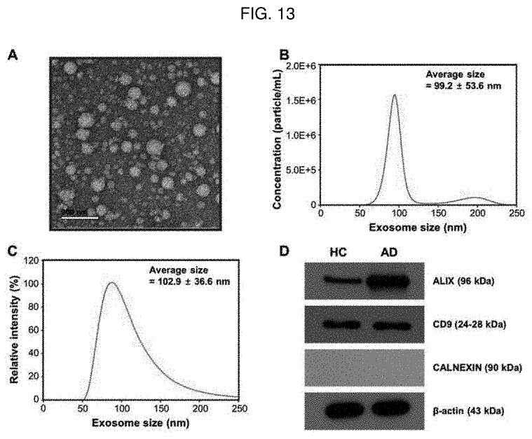

Characterization of the exosomes was performed to determine whether or not the exosomes were properly isolated from the serum samples. The morphology of the isolated exosomes was observed using TEM, nanoparticle tracking analysis (NTA), and dynamic light scattering (DLS). The result showed that the exosomes are spheres having a size ranging from 30 to 150 nm (FIGS. 13A to 13C). Expression of exosome-specific markers (CD9 and ALIX) on the exosome membrane was identified by Western blotting (FIG. 13D).

Then, miRNA was isolated from the exosomes using a total exosome RNA and protein isolation kit in accordance with the manual (Methods in support information). The difference in the expression level of three target biomarkers between the AD group and the HC group was determined using conventional standard qRT-PCR and the plasmonic biosensor of the present invention. Statistical significance was determined using the Mann-Whitney U test.

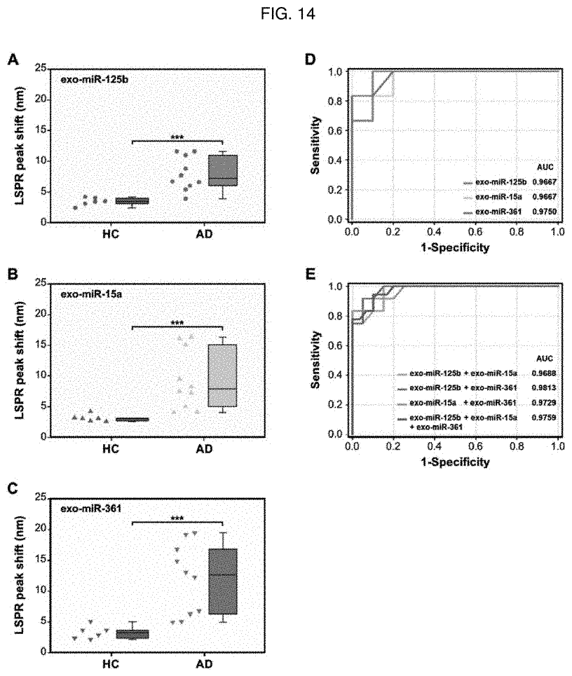

As a result, as can be seen from FIG. 14, the average LSPR redshift λ max of the AD group was 2.09 (for exo-miR-125b, p=0.0014), 2.53 (for exo-miR-15a, p=0.0014) and 3.85 (for exo-miR-361, p=0.0012 times higher). These results indicate that the LSPR redshift of three exo-miRs can be used to accurately distinguish AD patients from the HCs (FIGS. 14A to 14C).

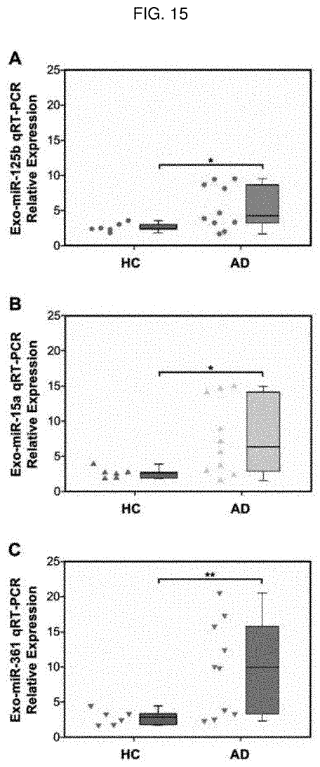

Similarly, the relative expression levels of the three biomarkers measured by qRT-PCR also showed a similar tendency to those of the biosensor of the present invention. However, it was confirmed that the statistical significance was low compared to the biosensor of the present invention (p<0.05) (FIG. 15).

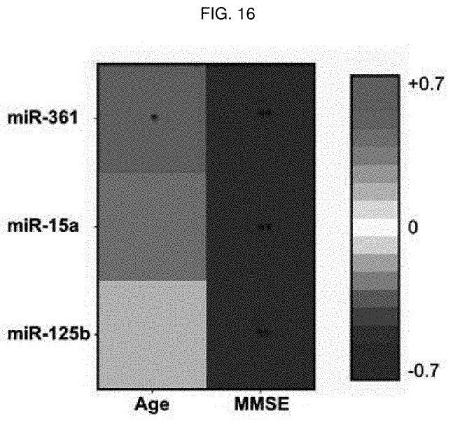

Finally, correlations between the relative expression levels of the three exo-miRs and AD-related parameters were determined. The three biomarkers showed a positive correlation with age and a strong negative correlation with the MMSE (mini-mental state examination) score (FIG. 16). Then, the diagnostic efficacy was predicted by analyzing the curve value of the ROC (receiver operating characteristic) of each biomarker. ROC analysis is a standard statistical approach for evaluating the performance of diagnostic tests, such as the sensitivity, selectivity, and accuracy of a sensor. The area under curve (AUC) was a value close to 1.0, meaning an accurate clinical prediction, and was used to quantify the diagnostic accuracy of the sensor.

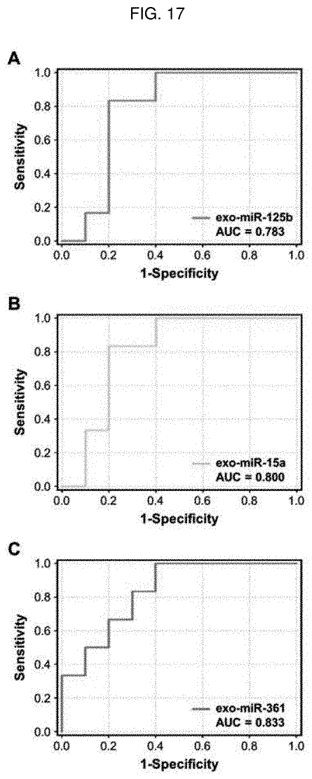

As a result, the AUC value of the DAPA-based plasmonic biosensor of the present invention was higher than 0.96 for each of the three biomarkers (FIG. 14D), but the conventional qRT-PCR method was only able to yield smaller AUC values ranging from 0.78 to 0.84 (FIG. 17).

In addition, the biosensor of the present invention can successfully discriminate between Alzheimer's disease patients and HCs with an average sensitivity of 83.33%, selectivity of 90.00%, and accuracy of 97.83% using the respective exo-miR biomarkers. Among them, exo-miR-361 (AUC: 0.9750) was determined to be the optimal single biomarker for AD diagnosis (Table 4).

TABLE 4
Summary of diagnostic performance using single
DAPA-based label-free plasmonic biosensor

| | Cut-off | Sensi- | Selec- | Accu- | |
|---|---|---|---|---|---|
| | value | tivity | tivity | racy | |
| Biomarkers | (nm) | (%) | (%) | (%) | AUC |
| Exo-miR-125b | ≥4.10 | 83.33 | 90.00 | 97.83 | 0.9667 |
| Exo-miR-15a | ≥4.05 | 83.33 | 90.00 | 97.83 | 0.9667 |
| Exo-miR-361 | ≥5.05 | 83.33 | 90.00 | 97.83 | 0.9750 |
| Exo-miR-125b + | ≥4.05 | 83.33 | 90.00 | 97.83 | 0.9688 |
| Exo-miR-15a | | | | | |
| Exo-miR-125b + | ≥4.60 | 91.67 | 95.00 | 99.52 | 0.9813 |
| Exo-miR-361 | | | | | |
| Exo-miR-15a + | ≥4.60 | 91.67 | 90.00 | 99.00 | 0.9729 |
| Exo-miR-361 | | | | | |
| Exo-miR-125b + | ≥4.60 | 94.44 | 90.00 | 99.35 | 0.9759 |
| Exo-miR-15a + | | | | | |
| Exo-miR-361 | | | | | |

*Sensitivity = true positive value/total positive value, selectivity = true negative value/total negative value, accuracy = total true value/total value

Evaluating a combination of biomarkers rather than a single biomarker is more effective in identifying subjects having increased risk of Alzheimer's disease among cognitively normal subjects due to the complexity of Alzheimer's disease pathogenesis. Therefore, a combination of exo-miR biomarkers enabling accurate diagnosis of Alzheimer's disease was searched for.

As a result, as can be seen from FIG. 14E, the combination of exo-miR-125b and exo-miR-361 showed the best AD diagnostic performance, more specifically, AUC of 0.9813, sensitivity of 91.67%, selectivity of 95.00%, and accuracy of 99.52% (FIG. 14E).

These results suggest that Alzheimer's disease patients can be identified with an accuracy of 97.83% or more by quantifying the expression levels of exo-miR-125b, exo-miR-15a, and exo-miR-361 in clinical samples using the DAPA-based plasmonic biosensor of the present invention.

In conclusion, the DAPA-based plasmonic biosensor of the present invention is capable of detecting miRNAs with an ultrahigh sensitivity at an attomolar level because the narrow nanogap (2 nm) of DAPA creates a plasmonic coupling effect, and is capable of identifying even single-nucleotide differences between miRNAs due to hybridization between the LNA probe and the target miRNA occurring on the DAPA surface.

In particular, the clinical relevance of the DAPA-based sensor to three different types of exo-miRs (exo-miR-125b, exo-miR-15a, and exo-miR-361) in clinical sera was verified, and these exo-miRNAs were found to be suitable for clinical application based on the clinical sensitivity, selectivity, and accuracy of the sensor. Among them, the combination of exo-miR-125b and exo-miR-361 showed the best diagnostic performance, distinguishing Alzheimer's disease patients from HC patients with an accuracy of 99.52%.

These results suggest that the DAPA-based plasmonic biosensor profiling exo-miRNA according to the present invention can be a promising candidate for early diagnosis of dementia and prediction of the progression of dementia by profiling of exosomal miRNAs in clinical practice.

As apparent from the foregoing, according to an embodiment of the present invention, the DAPA-based plasmonic biosensor enables multiple label-free detection of pathogenesis markers present in blood, for example, exosome-derived miRNA, with high selectivity and high sensitivity, and thus is effectively utilized in the diagnostic and clinical application of miRNA-related diseases.

Although specific configurations of the present invention have been described in detail, those skilled in the art will appreciate that this detailed description is provided as preferred embodiments for illustrative purposes and should not be construed as limiting the scope of the present invention. Therefore, the substantial scope of the present invention is defined by the accompanying filed claims and equivalents thereto.

What is claimed is:

1. A single metal-DAPA comprising:
at least one metal nanosphere conjugated with one single-stranded DNA (1ssDNA); and
at least one metal nanosphere conjugated with two single-stranded DNAs (2ssDNA) complementary thereto,
wherein the metal nanospheres are bridged to one another and the single metal-DAPA comprises a nanogap greater than 0 and not greater than 2 nm.

2. The single metal-DAPA according to claim 1, wherein the DAPA comprises the metal nanosphere conjugated with one single-stranded DNA (1ssDNA) and the metal nanosphere conjugated with two single-stranded DNAs (2ssDNA) complementary thereto at a ratio of 2:1.

3. The single metal-DAPA according to claim 1, wherein the metal comprises any one selected from the group consisting of gold (Au), copper (Cu), platinum (Pt) and palladium (Pd).

4. The single metal-DAPA according to claim 1, wherein the single metal-DAPA is used for a biosensor.

5. A single DAPA-based label-free nanoplasmonic biosensor, comprising:
a substrate;
a metal-DAPA (DNA-assembled advanced plasmonic architecture) fixed to the substrate, the metal-DAPA conjugated with a capture probe specifically binding to a target biomarker; and
a measuring device configured to measure localized surface plasmon resonance in the metal-DAPA.

6. The single DAPA-based label-free nanoplasmonic biosensor according to claim 5, wherein the metal-DAPA comprises three metal nanospheres conjugated with single-stranded DNA and bridged to one another.

7. The single DAPA-based label-free nanoplasmonic biosensor according to claim 6, wherein the DAPA comprises at least one metal nanosphere conjugated with one single-stranded DNA (1ssDNA) and at least one metal nanosphere conjugated with two single-stranded DNAs (2ssDNA) complementary thereto at a ratio of 2:1.

8. The single DAPA-based label-free nanoplasmonic biosensor according to claim 6, wherein the DNA has a length of 75 bp to 150 bp.

9. The single DAPA-based label-free nanoplasmonic biosensor according to claim 5, wherein the metal-DAPA comprises a nanogap.

10. The single DAPA-based label-free nanoplasmonic biosensor according to claim 9, wherein the nanogap is greater than 0 nm and not greater than 2 nm.

11. The single DAPA-based label-free nanoplasmonic biosensor according to claim 5, wherein the metal comprises any one selected from the group consisting of gold (Au), copper (Cu), platinum (Pt) and palladium (Pd).

12. The single DAPA-based label-free nanoplasmonic biosensor according to claim 5, wherein the target biomarker is DNA, miRNA or peptide.

13. The single DAPA-based label-free nanoplasmonic biosensor according to claim 12, wherein the miRNA is derived from an exosome.

14. The single DAPA-based label-free nanoplasmonic biosensor according to claim 13, wherein the miRNA is exo-miR125b, exo-miR15a, exo-miR361, or a combination thereof.

15. The single DAPA-based label-free nanoplasmonic biosensor according to claim 14, wherein the biosensor detects exosome-derived miRNA proteins by measuring a change in Rayleigh scattering spectrum caused by specific binding of exosome-derived miRNA.

16. The single DAPA-based label-free nanoplasmonic biosensor according to claim 5, wherein the capture probe comprises DNA or LNA.

17. A method of detecting exosome-derived miRNA comprising treating the biosensor according to claim 5 with a biomarker mixture.

18. The method according to claim 17, wherein the biomarker mixture is blood or agglutinin-free serum.

19. The method according to claim 18, wherein the biomarker mixture comprises exosome-derived miRNA.

20. A method of diagnosing Alzheimer's disease comprising:
treating the biosensor according to claim 5 with a biomarker mixture; and
treating the biosensor with an exosome-derived miRNA detection probe.

21. The method according to claim 20, wherein the detection probe comprises DNA or LNA.

22. A method of preparing a single DAPA-based nanoplasmonic biosensor, the method comprising:
(a) hybridizing single-stranded DNA-metal nanospheres with single-stranded DNA-metal nanospheres having a sequence complementary thereto at a ratio of 2:1 to obtain metal-DAPA seeds;
(b) coating the metal-DAPA seeds with a predetermined material;
(c) crystallizing the coated metal-DAPA seeds by treatment with a metal precursor and a reducing agent;
(d) fixing the metal-DAPA to a substrate; and
(e) conjugating the metal-DAPA with a capture probe specifically binding to an isolated target biomarker.

23. The method according to claim 22, wherein the metal-DAPA seeds are coated with polyethylene glycol (PEG).

24. The method according to claim 18, further comprising coating the substrate with (3-mercaptopropyl)trimethoxysilane (MPTES) before step (d).

25. The method according to claim 18, wherein the crystallization is performed at a pH of 5.

26. A method of preparing a single metal-DAPA, the method comprising:
(a) hybridizing one single-stranded DNA (1ssDNA)-metal nanospheres with two single-stranded DNAs (2ssDNA)-metal nanospheres having a sequence complementary thereto at a ratio of 2:1 to obtain metal-DAPA seeds;
(b) coating the metal-DAPA seeds with a predetermined material; and
(c) crystallizing the coated metal-DAPA seeds by treatment with a metal precursor and a reducing agent.
